(19) United States
(12) Patent Application Publication
(10) Pub. No.: US 2024/0074653 A1
(43) Pub. Date: Mar. 7, 2024

(54) SYSTEM AND A METHOD FOR COLOR-BLIND PERSON TO DISTINGUISH COLORS IN A SURROUNDING

(71) Applicant: Mirza Faizan, Irving, TX (US)

(72) Inventors: Mirza Faizan, Irving, TX (US); Isha Agrawal, Plano, TX (US); Tarik Syed, Dallas, TX (US); Nakshatra Piduri, Plano, TX (US); Advaita Piduri, Plano, TX (US); Madhalasa Iyer, Plano, TX (US); Mirza Rizwan, Patna (IN); Nithyashri Ramesh, Irving, TX (US); Hisham Ahmad, Allen, TX (US); Abdullah Kabeer, Plano, TX (US); Mishaal Qureshi, Frisco, TX (US); Abdullah Hasani, Murphy, TX (US); Zaynab Khan, Murphy, TX (US)

(21) Appl. No.: 17/900,922

(22) Filed: Sep. 1, 2022

(51) Int. Cl. A61B 3/06; G02C 5/00; G06T 11/00

(52) U.S. Cl. CPC A61B 3/066; G02C 5/001; G06T 11/001; G02C 2202/10

(57) ABSTRACT

The present disclosure envisages a system for facilitating a color-blind person to accurately distinguish between certain shades of color in a surrounding. The system comprises an eye frame configured to obtain a plurality of information corresponding to each object present in the surrounding and a handheld device configured to cooperate with said eye frame through a communication network to receive the plurality of information corresponding to each object. The handheld device is configured to process the plurality of information for facilitating a color-blind person to accurately distinguish between certain shades of color in the surrounding.

### FIELD

The present disclosure relates to the field of devices helpful for color blind people. More specifically, the invention belongs to the field of devices that facilitate a color-blind person to accurately distinguish between certain shades of color in a surrounding.

### BACKGROUND

Color blindness is an ophthalmic disorder related to the eyes of a person and affects the visual sensitivity of a person. The disorder leads to failure in differentiation between various colors present in the surrounding. To emphasize, the person with a disorder cannot discriminate between various visible color wavelengths that a normal person may do with naked eyes.

There are several types of color blindness addressed in medical science. Individuals with a weak first color are less sensitive to red light than normal individuals and thus suffer from darkening at the red spectral edge. The second weak color individual has a mutant form of a green pigment that is shifted towards the red spectral edge, resulting in reduced sensitivity to the green spectral region. Similar to the first color weak, the second color weak is not good at identifying small differences in hues in the red, orange, yellow, and green spectral regions.

Traditionally, several methods and devices were proposed for correcting human color blindness. Some commonly known methods for correcting the lack of visual color discrimination include differential coloration, filtration, and spectral transmission between simultaneously used lenses.

Another commonly known visibility correction method is eye surgery using ophthalmic devices. The ophthalmic devices tend to be large, bulky, and expensive devices and are typically used exclusively in doctor's offices. Thus, patients may be required to make an appointment with an optometrist and visit the doctor for any treatment. The worsened condition may require even more drastic therapies or procedures to address when it could have been more easily alleviated had the patient been timely diagnosed or treated. Furthermore, the large and bulky nature of most ophthalmic devices forces patients to be placed in an uncomfortable position for a large amount of time, which in turn may actually increase the risks of misdiagnosis and patient error. These surgeries may also lead to scars on the patient's face and may permanently leave a mark on the face.

There is, therefore, felt a need for a system and a method that facilitate a color-blind person to accurately distinguish between certain shades of color in a surrounding. The present invention further facilitates avoiding appointments, hectic surgeries, and the use of large machines by doctors.

### OBJECTS

Some of the objects of the present disclosure, which at least one embodiment herein satisfies, are as follows:

An object of the present disclosure is to provide a system and a method that facilitate a color-blind person to accurately distinguish between certain shades of color in a surrounding.

Another object of the present disclosure is to provide a lightweight device to correct color blindness.

Still another object of the present disclosure is to provide a system and a method that facilitates avoiding long queues and appointments at doctors' offices.

Yet another object of the present disclosure is to provide a system and a method that prevents hectic surgeries.

A further object of the present disclosure is to provide a system and a method that avoids the usage of large machines by doctors.

A still further object of the present disclosure is to provide a system and a method that use lightweight glasses.

An object of the present disclosure is to provide a system and a method that provide easy charging of the glasses.

Another object of the present disclosure is to provide a system and a method that is easy to use.

A further object of the present disclosure is to provide a system and a method that is economical for both doctors and patients.

Other objects and advantages of the present disclosure will be more apparent from the following description, which is not intended to limit the scope of the present disclosure.

### SUMMARY

The present invention envisages a system for facilitating a color-blind person to accurately distinguish between certain shades of color in a surrounding. The system comprises an eye frame configured to obtain a plurality of information corresponding to each object present in the surrounding.

The eye frame comprises a first layer configured to obtain color information corresponding to each object present in the surrounding.

The eye frame further comprises an image capturing module configured to capture at least one image or at least one video of the surrounding.

The system of the present invention further comprises a handheld device configured to cooperate with the eye frame through a communication network to receive the plurality of information corresponding to each object.

The handheld device is configured to process the plurality of information for facilitating a color-blind person to accurately distinguish between certain shades of color in the surrounding.

In an embodiment, the handheld device includes an image processing module configured to process at least one image or at least one video to provide actual color information corresponding to each object.

In another embodiment, the handheld device includes a receiving module configured to receive the color information and the actual color information corresponding to each object.

In still another embodiment, the handheld device includes a determining module configured to determine an ideal image corresponding to said objects.

In a further embodiment, the ideal image is obtained by calculating a difference between the color information obtained from the first layer and the actual color information obtained by the image capturing module.

In yet another embodiment, the eye frame further comprises a second layer configured to receive the ideal image of the objects.

In a further embodiment, the second layer is configured to display the ideal image of the objects to the color-blind person.

In a still further embodiment, the image capturing module is located at the first end of each leg of the eye frame.

A method for facilitating a color-blind person to accurately distinguish between certain shades of color in a surrounding, the method comprising steps of:
obtaining, by an eye frame, a plurality of information corresponding to each object present in the surrounding;
receiving, by a handheld device, the plurality of color information corresponding to each object; and
processing, by the handheld device, the plurality of color information for facilitating a color-blind person to accurately distinguish between certain shades of color in the surrounding.

In an embodiment, the processing includes determining an ideal image corresponding to objects, wherein the ideal image is obtained by calculating a difference between color information obtained from a first layer of the eye frame and actual color information obtained by at least one camera of the handheld device.

In another embodiment, the step of determining further comprises receiving the ideal image of the objects by a second layer present on the eye frame.

In still another embodiment, the step of receiving further comprises displaying the ideal image of the objects to the color-blind person.

These and other features, advantages, and objects of the various embodiments will be better understood with reference to the following drawings, specifications and claims.

### BRIEF DESCRIPTION OF THE ACCOMPANYING DRAWING

The accompanying drawings, which are included to provide a further understanding of the invention and are incorporated in and constitute a part of this specification, illustrate embodiments of the invention and together with the description serve to explain the principles of the invention.

The following figure depicts certain illustrative embodiment of the invention. This depicted embodiment is to be understood as illustrative of the invention and not as limiting in any way.

Referring particularly to the drawing for the purpose of illustration only and not limitation, there is illustrated:

A system and a method for facilitating a color-blind person to accurately distinguish between certain shades of color in a surrounding, is now described in accordance with preferred embodiment of the present disclosure, in which:

FIG. 1 illustrates an exemplary network environment for facilitating a color-blind person to accurately distinguish between certain shades of color in a surrounding, in accordance with an embodiment of the present disclosure;

FIG. 2 illustrates an exemplary functional block diagram of handheld device of FIG. 1;

FIG. 3 illustrates an exemplary diagram of eye frame of FIG. 1; and

FIG. 4 illustrates an exemplary method flow diagram for facilitating a color-blind person to accurately distinguish between certain shades of color in a surrounding, in accordance with an embodiment of the present disclosure.

### LIST OF REFERENCE NUMERALS

100—System
102—Eye frame
104—Handheld device
106—Communication network
108—Memory
110—Processor
112—Interface
202—Receiving module
204—Image processing module
206—Determining module
208—Other data
210—Configuration data
212—Device & application data
214—User related data
302—First layer
304—Image capturing module
306—Second layer

### DETAILED DESCRIPTION

Embodiments are provided so as to thoroughly and fully convey the scope of the present disclosure to the person skilled in the art. Numerous details are set forth relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

The following detailed description illustrates the invention by way of example, and not by way of limitation. This description will clearly enable one skilled in the art to make and use the invention and describes several embodiments, adaptions, variations, alternatives, and uses of the invention, including what we presently believe is the best mode of carrying out the invention.

The present invention envisages a system and a method for facilitating a color-blind person to accurately distinguish between certain shades of color in a surrounding. The present invention further facilitates avoiding appointments, hectic surgeries, and the use of large machines by doctors.

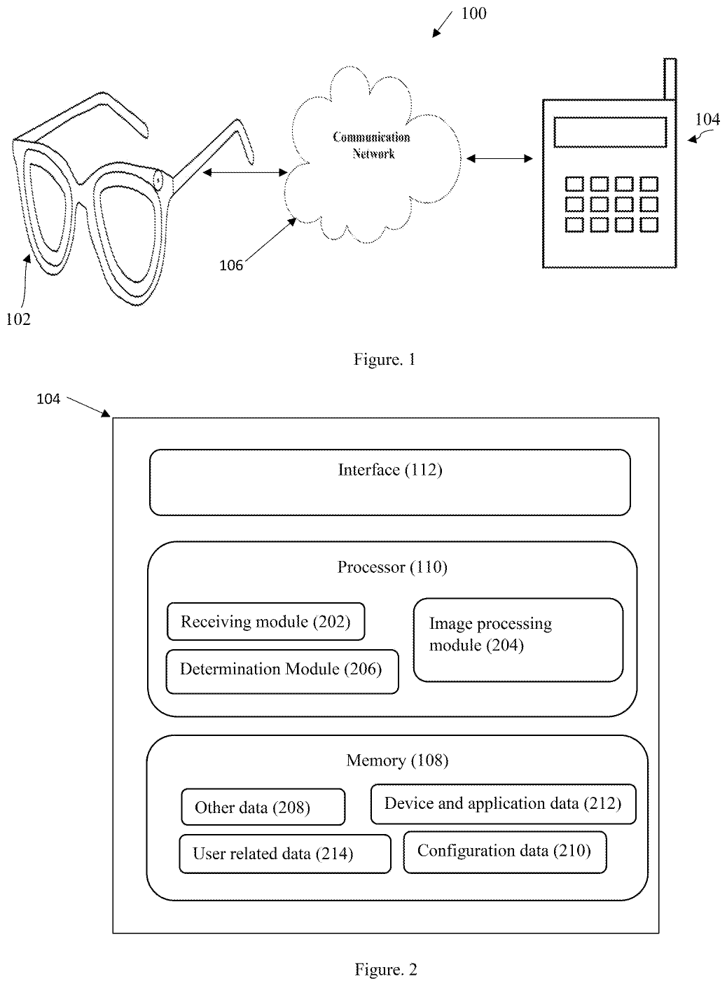

The present invention will now be described with reference to FIG. 1 of the preferred embodiment of the present invention.

The present invention envisages a system 100 for facilitating a color-blind person to accurately distinguish between certain shades of color in a surrounding. System 100 comprises an eye frame 102 and a handheld device 104.

The system 100 is not limited to only components 102 and 104 as communication between 102 and 104 is provided by means of a communication network 106. Network connectivity can be wired or wireless between 102 and 104.

However, wireless connectivity is preferable for the convenience of the user. Examples of wireless connectivity between 102 and 104 can be selected from Bluetooth, infrared and other types that support devices in proximity to connect.

In an embodiment, the communication network 106 may include, but is not limited to, a direct interconnection, an e-commerce network, a Peer-to-Peer (P2P) network, Local Area Network (LAN), Wide Area Network (WAN), wireless network (for example, using Wireless Application Protocol), Internet, Wi-Fi and the like.

The eye frame 102 can also communicate to the handheld device 104 via a cloud server where proximity between 102 and 104 becomes a constraint. For example, in cases where a doctor or a caretaker remotely wants to control a patient's eye frame 102.

The main function of the eye frame 102 is to obtain a plurality of information corresponding to each object present in the surrounding. For example, the information may relate to color information of an object present in the surrounding of the patient or user.

In a preferred embodiment, the user may choose glasses that are simple in design, i.e., a frame, or could be made according to the user's prescription.

Further, eye frame 102 is configured to obtain color information corresponding to each object present in the surrounding. The eye frame 102 is configured to capture at least one image or at least one video of the surrounding.

In an embodiment, the eye frame may include 2 high-definition cameras to capture images of the surrounding for clarity.

In another embodiment, the camera(s) is located at the first end of each leg of the eye frame 102.

In still another embodiment, the image of the surrounding provides actual color information corresponding to each object.

In a preferred embodiment, the eye frame 102 can be charged using a frame case. The eye frame 102 in such a scenario includes a contact point (not shown in the figure) which is connected to the magnetic contact charging port of the frame case.

In another embodiment, eye frame 102 contains inductive coils in the front frame and batteries in the side handles for inductive charging purposes.

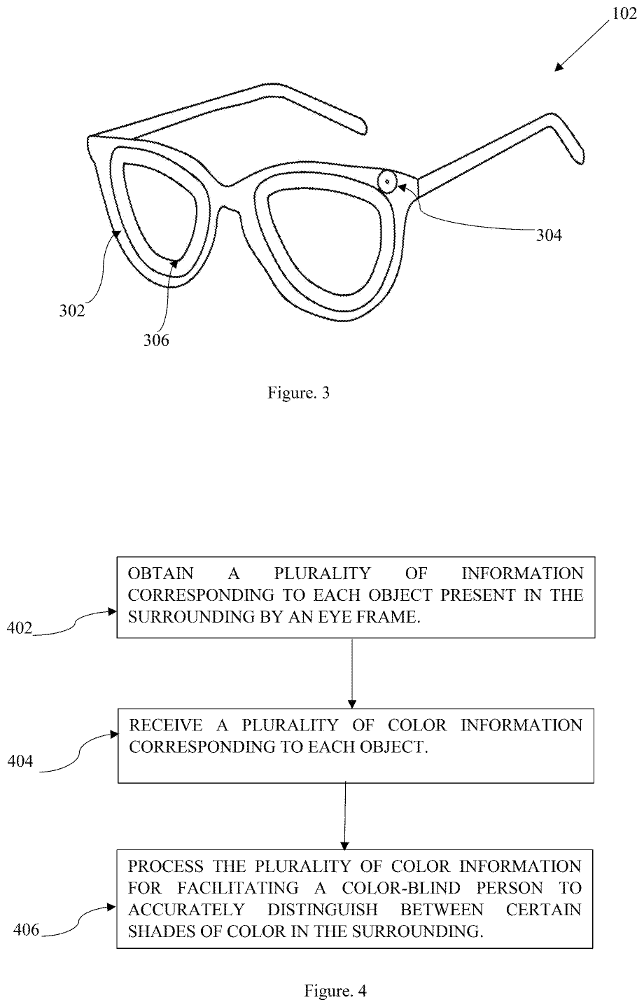

FIGS. 2 and 3 are now described for a detailed view of a handheld device 104 and the eye frame 102 of FIG. 1.

The system 100 further comprises the handheld device 104 configured to cooperate with the eye frame 102 through the communication network 106 to receive the plurality of information corresponding to each object.

Precisely, the handheld device 104 receives color information corresponding to each object present in the surrounding and at least one image of the surrounding.

The handheld device 104 is further configured to process the plurality of information for facilitating a color-blind person to accurately distinguish between certain shades of color in the surrounding.

The handheld device 104 may include but is not limited to, a laptop, a desktop computer, a notebook, a smartphone, IoT devices, smart car infotainment system or smart home application devices, a tablet, a server, and any other computing devices. A person skilled in the art would understand that any other devices, not mentioned explicitly, may also be used as the handheld device in the present disclosure.

In a preferred embodiment, the eye frame 102 includes a wireless chip that connects to the user's handheld device using a wireless link, for example, Bluetooth preferably.

In an embodiment, the eye frame 102 can perform charging after connecting with the handheld device 104.

In another embodiment, the eye frame 102 can be used for 8 to 10 hours straight without charging. The eye frame 102 requires charging 6 hours for functioning.

The handheld device 104 is implemented by a processor 110 that may be a general-purpose processor, a digital signal processor, an application-specific integrated circuit, a field-programmable gate array or another programmable logic device, a discrete gate or transistor logic device, or a discrete hardware component, and can implement or perform the methods, steps, and logical block diagrams disclosed in the embodiments of this invention.

The general-purpose processor may be a microprocessor or any conventional processor or the like. The steps of the method disclosed with reference to the embodiments of this application may be directly performed by a hardware processor, or may be performed by using a combination of hardware in the processor and a software unit. The handheld device 104 includes an I/O interface 112, a memory 108, and the processor 110. The I/O interface 112 may be configured to receive data from the eye frame 102

The data from the I/O interface 112 may be stored in memory 108. The memory 108 may be communicatively coupled to the processor 110 that executes all the modules.

The memory 108 may also store processor instructions which may cause the processor 110 to execute the instructions for managing continuous execution of the modules.

In an embodiment, the processor 110 is configured to employ at least one of the machine learning techniques to facilitate a color-blind person to accurately distinguish between certain shades of color in the surrounding.

In another embodiment, the machine learning techniques are selected from the group consisting of predictive analysis techniques and time series analysis techniques.

In yet another embodiment, the machine learning algorithms employed include, but are not limited to, Naïve Bayes, decision trees, linear regression, neural networks, random forest, and the like.

The processor 110 includes a receiving module 202, a determining module 206, and an image processing module 204.

The image processing module 204 is configured to process at least one image or at least one video to provide actual color information corresponding to each object.

The receiving module 202 is configured to receive the color information and the actual color information corresponding to each object and the determining module 206 is configured to determine an ideal image corresponding to the objects.

The ideal image is obtained by calculating a difference between the color information obtained from a first layer 302 of the eye frame 102 and the actual color information obtained by the image capturing module 304.

In an embodiment, the receiving module 202 is configured to receive video sequences from the camera of the eye frame 102. The received video segments are segmented to extract a clear segment of the image. The determining module 206, in such a scenario, employs image recognition and segmentation techniques.

In an embodiment, the eye frame 102 further comprises a second layer 306 configured to receive the ideal image of the objects. This ideal image of the objects is displayed on the second layer 306 of the eye frame 102 which is visible to the color-blind person or user.

The present invention has two layers present on the eye frame 102, known as, an active 302 and a passive layer 306. The passive layer is on the top layer of the glasses (behind the active layer) and it's tinted to reflect colors in the environment. The present invention requires an internet connection, the passive layer of just tinted glasses would be beneficial when a colorblind individual is in an environment that lacks an internet connection. In such a situation, the color-blind individual cannot sense contrast but the glass could resort to the normal colorblind glasses.

In an embodiment, the eye frame 102 enhances contrast through the use of hetero-chromic filters. In a working example, the lens of the eye frame 102 is tinted pale green while the other lens is tinted to be a slightly darker yellow. This results in the creation of color-retinal rivalry between the two eyes to encourage better visual contrast and provide improved vision quality, especially at night.

In another preferred embodiment, various type of data is stored in the handheld device 104 that may include, for example, configuration data 210, device and application data 212, user-related data 214, and other data 208.

The configuration data 210 may include configuration information associated with the monitoring device 104. The configuration data 210 may contain a list of registered users and user devices. In an embodiment, the list is updated when a new user device is registered.

The device and application data may include device configuration information such as device identification number, device type, and supported application identification number, used application details such as, comprises application identifier application type, application state, and application restriction.

The user-related data 214 may include registered user information such as user identification number, date, time, the location associated with the devices.

The other data 208 may store data, including temporary data and temporary files, generated by modules for performing the various functions of the monitoring device 104.

FIG. 4 will now be described that illustrates a method 400 for facilitating a color-blind person to accurately distinguish between certain shades of color in a surrounding.

The present invention envisages a method 400 for facilitating a color-blind person to accurately distinguish between certain shades of color in a surrounding.

Method 400 includes obtaining 402 a plurality of information corresponding to each object present in the surrounding by an eye frame 102. The plurality of color information corresponding to each object is received 404 by a handheld device 104.

The method 400 further includes the step of processing 406, by the handheld device 104, the plurality of color information for facilitating a color-blind person to accurately distinguish between certain shades of color in the surrounding.

The processing 406 includes determining an ideal image corresponding to objects, wherein the ideal image is obtained by calculating a difference between color information obtained from a first layer 302 of said eye frame and actual color information obtained by image capturing module 304 of the handheld device 104.

The step of determining further comprises receiving the ideal image of the objects by a second layer 306 present on the eye frame and displaying the ideal image of the objects to the color-blind person.

According to the system and method disclosed herein, a system and method in accordance with the present invention provide numerous benefits. It provides a useful, novel, and non-trivial solution to the problem of presenting, capturing, and modifying images for helping color blind people.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

The foregoing description of the embodiments has been provided for purposes of illustration and not intended to limit the scope of the present disclosure. Individual components of a particular embodiment are generally not limited to that particular embodiment but are interchangeable. Such variations are not to be regarded as a departure from the present disclosure, and all such modifications are considered to be within the scope of the present disclosure.

### TECHNICAL ADVANCEMENTS

The present disclosure described herein above has several technical advantages including, but not limited to, the realization of a system and a method to facilitate a color-blind person:
to accurately distinguish between certain shades of color in a surrounding;
with a light weight device to correct color blindness;
for avoiding long queues and appointments at doctors' office;
to prevent hectic surgeries;
to avoid usage of large machines by the doctors;
to use lightweight glasses;
to provide easy charging of the glasses;
easy to use the system;
with an economical system for both doctors and patients that have simple construction; and
with a cost-effective system.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The use of the expression “at least” or “at least one” suggests the use of one or more elements or ingredients or quantities, as the use may be in the embodiment of the disclosure to achieve one or more of the desired objects or results.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

Therefore, while the embodiments herein have been described in terms of preferred embodiments, those skilled in the art will recognize that the embodiments herein can be practiced with modification within the spirit and scope of the embodiments as described herein.

To the preferred embodiments of the invention, a skilled person, to satisfy contingent requirements, may make modifications, adaptations, and replacements of elements with others functionally equivalent, without departing from the scope of the following claims. Each of the characteristics described as belonging to a possible preferred embodiment can be achieved independently from the other preferred embodiments described

The above description is considered that of the preferred embodiment(s) only. Modifications of these embodiments will occur to those skilled in the art and to those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

What is claimed is:

1. A system for facilitating a color-blind person to accurately distinguish between certain shades of color in a surrounding, said system comprising:
an eye frame configured to obtain a plurality of information corresponding to each object present in said surrounding, said eye frame comprising:
a first layer configured to obtain a color information corresponding to each object present in said surrounding; and
an image capturing module configured to capture at least one image or at least one video of said surrounding; and
a handheld device configured to:
cooperate with said eye frame through a communication network to receive said plurality of information corresponding to each object; and
process said plurality of information to facilitate a color-blind person to accurately distinguish between certain shades of color in said surrounding.

2. The system as claimed in claim 1, wherein said handheld device includes an image processing module configured to process said at least one image or at least one video to provide an actual color information corresponding to each object.

3. The system as claimed in claim 1, wherein said handheld device includes:
a receiving module configured to receive said color information and said actual color information corresponding to said each object; and
a determining module configured to determine an ideal image corresponding to said objects, wherein the ideal image is obtained by calculating a difference between said color information obtained from said first layer and said actual color information obtained by said image capturing module.

4. The system as claimed in claim 3, wherein said eye frame further comprises a second layer configured to receive said ideal image of said objects.

5. The system as claimed in claim 4, wherein said second layer is configured to display said ideal image of said objects to said color-blind person.

6. The system as claimed in claim 1, wherein said image capturing module is located at a first end of each leg of said eye frame.

7. A method for facilitating a color-blind person to accurately distinguish between certain shades of color in a surrounding, said method comprising steps of:
obtaining, by an eye frame, a plurality of information corresponding to each object present in said surrounding;
receiving, by a handheld device, said plurality of color information corresponding to said each object; and
processing, by said handheld device, said plurality of color information for facilitating a color-blind person to accurately distinguish between certain shades of color in said surrounding.

8. The method as claimed in claim 7, wherein said processing includes determining an ideal image corresponding to objects, wherein the ideal image is obtained by calculating a difference between a color information obtained from a first layer of said eye frame and an actual color information obtained by at least one image capturing module of said handheld device.

9. The method as claimed in claim 8, wherein step of determining further comprises receiving said ideal image of said objects by a second layer present on said eye frame.

10. The method as claimed in claim 9, wherein step of receiving further comprises displaying said ideal image of said objects to said color-blind person.
